(19) United States
(12) Patent Application Publication
(10) Pub. No.: US 2008/0287876 A1
(43) Pub. Date: Nov. 20, 2008

(54) Intravenous catheter introducing device

(76) Inventors: Ming-Jeng Shue, Taichung City (TW); Phillip Shue, Taichung City (TW); Deborah Huang, Taichung City (TW)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/218,887

(22) Filed: Jul. 18, 2008

Related U.S. Application Data
(63) Continuation-in-part of application No. 11/652,281, filed on Jan. 10, 2007.

(30) Foreign Application Priority Data
Jun. 16, 2006 (TW) 095121678
Jan. 17, 2008 (TW) 097101762

(51) Int. Cl. A61M 5/32; A61M 5/31

(52) U.S. Cl. 604/164.08; 604/168.01

(57) ABSTRACT

An intravenous catheter introducing device includes a barrel, a tubular grip member disposed to hold a tubular member and a needle seat to permit a tip end of a needle cannula to extend forwardly of the barrel, a tubular plunger movable along a passage of the barrel, and a tubular receptacle retained in the plunger. Forward movement of the plunger permits engagement of a socket end of the receptacle with the rear plug portion of the tubular member and release of the grip member from the barrel such that the needle cannula can be retracted into the plunger. The tubular member has a pre-chamber so that flashback blood flowing thereinto is visible.

### CROSS-REFERENCE TO RELATED APPLICATION

This application claims priority of Taiwanese Application No. 097101762, filed on Jan. 17, 2008.

This application is a continuation-in-part of U.S. patent application Ser. No. 11/652,281, which was filed on Jan. 10, 2007, and which claims priority of Taiwanese Application No. 095121678, filed on Jun. 16, 2006. The contents of the above referenced applications are herein incorporated by reference in their entirety.

### BACKGROUND OF THE INVENTION

1. Field of the Invention

This invention relates to an intravenous catheter introducing device, more particularly to an intravenous catheter introducing device with a tubular plunger which is operable to draw blood for facilitating a catheter introducing operation, and a needle cannula which is retractable into the tubular plunger for safe disposal.

2. Description of the Related Art

Intravenous catheter introducing devices are generally used to administer a medication fluid into or to draw blood from a patient's vein. Referring to FIG. 1, a conventional intravenous catheter introducing device 9 is shown to include a tubular needle seat 91 with a hub end 911, a needle cannula 94 secured to the hub end 911, a catheter hub 92 sleeved on the needle seat 91, and a flexible tubular catheter 93 secured to the catheter hub 92. In use, the catheter 93 and the needle cannula 94 are inserted into the patient's vein by a health care worker by piercing the patient's vein with a sharp tip of the needle cannula 94 which projects outwardly of the catheter 93. The health care worker then withdraws the needle cannula 94 from the catheter 93 with one hand and, at the same time, applies pressure to the patient's skin with the other hand, thereby leaving the catheter 93 in the patient's vein. Subsequently, a transfusion member (not shown) with the medication fluid or an empty barrel is connected to the catheter hub 92 for administering the medication fluid into the patient's vein or for drawing blood. At this time, the exposed sharp tip of the used needle cannula 94 may create a danger of an accidental needle stick.

Moreover, the conventional intravenous catheter introducing device 9 is specifically not suitable for patients whose blood pressure is not sufficient to permit flow of blood therethrough, such as an emergency case, aged people, and pediatrics patients, and for patients whose target vein is barely visible due to abundant adipose tissue, such as women and obese patients, since the health care worker will have difficulty determining whether the catheter 93 has been successfully introduced into the target vein, and may need to locate the vein by moving the needle cannula 94 in the skin of the patient, thereby complicating and prolonging the cannulation procedure and causing great discomfort to the patient.

Furthermore, although conventional self-retracting IV catheter introducers permit self-retraction of the used needle cannula into the syringe barrel after introduction of the catheter is completed, the blood in the barrel may be forced out of the barrel during the retraction of the used needle cannula, so that blood contamination may occur.

Therefore, in U.S. patent application Ser. No. 11/652,281, the applicants disclosed an intravenous catheter introducing device which includes a tubular plunger with outlets to communicate an accommodation compartment in the plunger with the ambient air so as to prevent blood from being forced out of the device during a needle retraction operation.

It is desirable to improve the aforesaid intravenous catheter introducing device to rapidly indicate whether a needle cannula has been inserted precisely into the vein of a patient for introduction of a tubular catheter.

### SUMMARY OF THE INVENTION

One object of the present invention is to provide an intravenous catheter introducing device which can rapidly indicate whether a needle cannula has been inserted precisely into the vein of a patient to introduce a tubular catheter, which can be operated easily and safely to retract the used needle cannula with one hand, and which can prevent the flashback blood from being forced out thereof.

According to this invention, the intravenous catheter introducing device includes:

a barrel having a surrounding barrel wall which surrounds an axis, and which defines a passage with rearward and forward openings, the surrounding barrel wall including a smaller-diameter portion which has an inner abutment surface facing rearwards, and a larger-diameter portion which has a retaining area that is spaced apart from the smaller-diameter portion;

a needle cannula having a tip end and a rear connecting end;

a tubular needle seat including a front hub portion which confronts the inner abutment surface so as to be engageable therewith, and a rear fitting portion which is configured to fix the rear connecting end;

a hollow tubular member which includes a front open end formed with the rear fitting portion to be in fluid communication with the rear connecting end, and tubular monitoring and gripped segments, the tubular monitoring segment defining a pre-chamber for flashback blood, the tubular member further including a rear plug portion which has an internal duct extending therethrough to be communicated with the pre-chamber;

a tubular grip member which, in a position of use, is disposed to hold, with a holding force, the gripped segment in a position of immovability along the axis relative to the retaining area;

a tubular plunger which is movable in the passage along the larger-diameter portion, the plunger having a front end wall which is movable to abut against the grip member, a rear end wall which extends outwardly of the rearward opening so as to be manually operable, and an intermediate surrounding wall which defines an accommodation compartment; and

a tubular receptacle which has a socket end, an air-permeable end, and a tubular wall segment confining a flashback chamber, the tubular receptacle being disposed to be retained in the accommodation compartment by a friction force, with the socket end and the air-permeable end respectively confronting the rear plug portion and the rear end wall, thereby establishing air communication between the internal duct and the flashback chamber,

such that, when the front end wall is brought to abut against the grip member, the socket end is engaged with the rear plug portion to thereby permit the front hub portion to be placed in a pre-disposal position,

such that, when the grip member is pushed forward by virtue of a forward movement of the front end wall against the holding force, the pre-disposal position facilitates release of the gripped segment from the grip member, thereby permitting an axial and rearward movement of the gripped segment relative to the retaining area, and

such that continued forward movement of the front end wall against the friction force results in movement of the needle seat, and movement of the tubular receptacle together therewith through the engagement of the rear plug portion with the socket end, towards the rear end wall by virtue of a prodding force so as to place the needle seat and the needle cannula in a disposal position, where the tip end of the needle cannula is retracted into the passage, and where the socket end is closer to the rear end wall than in the position of use.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a perspective view of a conventional IV catheter introducing device;

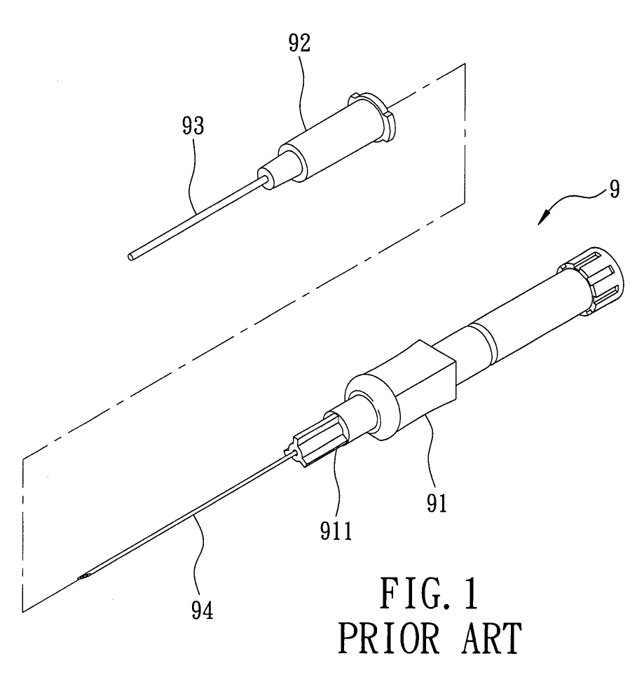

FIG. 2 is an exploded sectional view of a first preferred embodiment of an intravenous catheter introducing device according to this invention;

FIG. 3 is a sectional view of the first preferred embodiment in a pre-use position;

FIG. 4 is a fragmentary sectional view of a portion of the first preferred embodiment;

FIG. 5 is a sectional view of the first preferred embodiment in a position of use;

FIG. 6 is a sectional view of the first preferred embodiment in a pre-disposal position;

FIG. 7 is a sectional view of the first preferred embodiment to illustrate retraction of a needle cannula;

FIG. 8 is a sectional view of the first preferred embodiment in a retracted position;

FIG. 9 is a sectional view of the first preferred embodiment showing two outlets in a closed position for performing a blood drawing operation;

FIG. 10 is a sectional view of the second preferred embodiment of an intravenous catheter introducing device according to this invention;

FIG. 11 is a sectional view of the second preferred embodiment in a position of use;

FIG. 12 is a sectional view of the second preferred embodiment to illustrate retraction of a needle cannula;

FIG. 13 is a sectional view of the second preferred embodiment in a retracted position;

FIG. 14 is a sectional view of the second preferred embodiment showing an outlet in a closed position for performing a blood drawing operation;

FIG. 15 is a sectional view of the third preferred embodiment of an intravenous catheter introducing device according to this invention;

FIG. 16 is a sectional view of the third preferred embodiment in a retracted position;

FIG. 17 is a sectional view of the fourth preferred embodiment of an intravenous catheter introducing device according to this invention;

FIG. 18 is a sectional view of the fourth preferred embodiment in a retracted position;

FIG. 19 is a sectional view of the fourth preferred embodiment in a modified form;

FIG. 20 is a sectional view of the fifth preferred embodiment of an intravenous catheter introducing device according to this invention;

FIG. 21 is a sectional view of the sixth preferred embodiment of an intravenous catheter introducing device according to this invention;

FIG. 22 is a sectional view of the seventh preferred embodiment of an intravenous catheter introducing device according to this invention; and

FIG. 23 is a sectional view of the seventh preferred embodiment in a retracted position.

### DETAILED DESCRIPTION OF THE PREFERRED EMBODIMENTS

Before the present invention is described in greater detail, it should be noted that same reference numerals have been used to denote like elements throughout the specification.

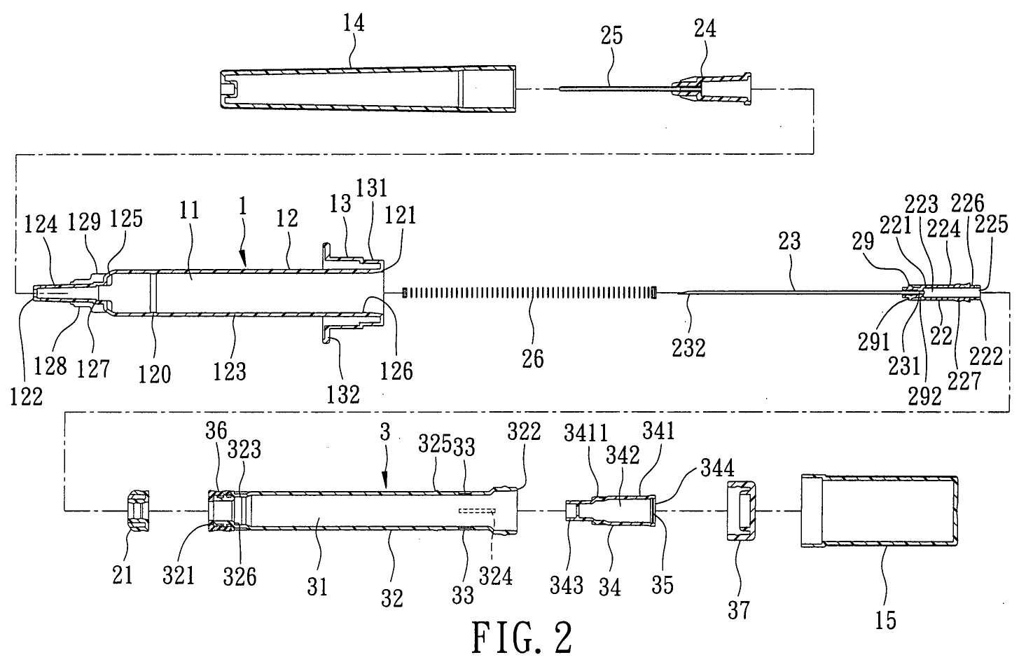

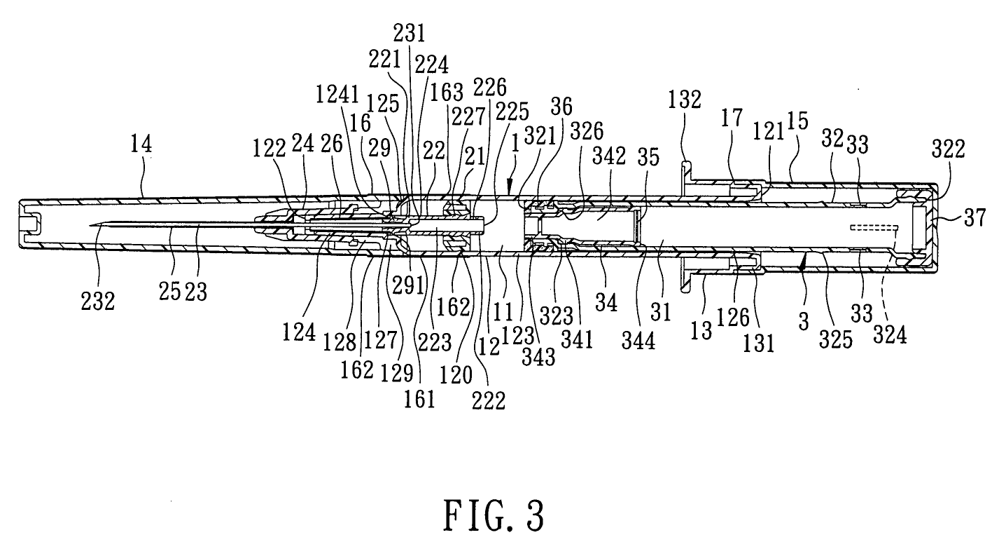

Referring to FIGS. 2 to 4, the first preferred embodiment of an intravenous catheter introducing device according to the present invention is shown to comprise a barrel 1, a needle cannula 23, a tubular needle seat 29, a hollow tubular member 22, a tubular grip member 21, a tubular plunger 3, a tubular receptacle 34, a coil spring 26, a catheter hub 24, a tubular catheter 25, and front and rear protectors 14, 15.

The barrel 1 has a surrounding barrel wall 12 which surrounds an axis, and which defines a passage 11 therein. The passage 11 has rearward and forward openings 121, 122 which are opposite to each other in a longitudinal direction. The surrounding barrel wall 12 includes a larger-diameter portion 123 and a smaller-diameter portion 124 which are disposed proximate to the rearward and forward openings 121, 122, respectively, and a shoulder 125 interposed therebetween. The smaller-diameter portion 124 has an inner abutment surface 127 which faces rearwards. The larger-diameter portion 123 has a retaining area 120 which is spaced apart from the smaller-diameter portion 124 in the longitudinal direction. The smaller-diameter portion 124 has first and second step segments 128, 129 proximate to the forward opening 122 and the shoulder 125, respectively.

In addition, the barrel 1 has a surrounding segment 13 which extends forwardly from the rearward opening 121 to terminate at a finger flange 132, and which surrounds the larger-diameter portion 123. The surrounding segment 13 has a narrow sleeved portion 131.

The needle cannula 23 has a tip end 232 and a rear connecting end 231 opposite to each other along the axis in the longitudinal direction.

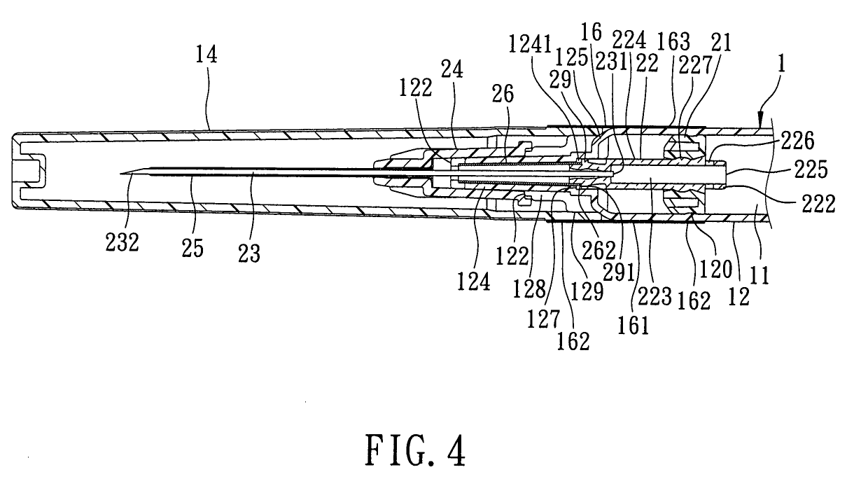

The tubular needle seat 29 includes a front hub portion 291 which confronts the inner abutment surface 127 so as to be engageable therewith, and a rear fitting portion 292 which is opposite to the front hub portion 291 in the longitudinal direction, and which is configured to fix the rear connecting end 231 of the needle cannula 23. Preferably, with reference to FIG. 4, the smaller-diameter portion 124 has a friction diminishing area 1241 which is disposed between the inner abutment surface 127 and the front hub portion 291.

The tubular member 22 is integrally formed with the tubular needle seat 29, and is made from a light transmissible material. The tubular member 22 includes a front open end 221 formed with the rear fitting portion 292 and in fluid communication with the rear connecting end 231, and tubular monitoring and gripped segments 224, 227 that are proximate to and distal from the front open end 221, respectively. The tubular monitoring segment 224 defines a pre-chamber 223 for flashback blood. The tubular member 22 further includes a rear plug portion 222 which extends from the gripped segment 227, which is distal from the front open end 221, and which has an internal duct 225 extending therethrough to be communicated with the pre-chamber 223.

The tubular grip member 21 is retained at the retaining area 120 in a position of use so as to hold the gripped segment 227 of the tubular member 22 in a position of immovability along the axis relative to the retaining area 120 by virtue of a holding force. Moreover, the rear plug portion 222 of the tubular member 22 is disposed rearwardly and outwardly of the grip member 21, and has a radial inlet 226 which extends radially to communicate the passage 11 with the pre-chamber 223.

The tubular plunger 3 is disposed to be movable in the passage 11 along the larger-diameter portion 123. The plunger 3 has an opened front end wall 321 which is movable to abut against the grip member 21, an opened rear end wall 322 which is disposed opposite to the front end wall 321, and which extends outwardly of the rearward opening 121 so as to be manually operable, and an intermediate surrounding wall 32 which is interposed between the front and rear end walls 321, 322, and which defines an accommodation compartment 31. The intermediate surrounding wall 32 has a plurality of ribs 324 which are formed on an outer surface thereof and adjacent to the rear end wall 322. The rear end wall 322 defines an access opening for insertion of the tubular receptacle 34 into the accommodation compartment 31. An end cap 37 is detachably mounted to the rear end wall 322 to close the access opening.

The plunger 3 further has a deformable annular sealing member 36 which is configured to surround the front end wall 321, and which is in air-tight sliding engagement with the larger-diameter portion 123 of the barrel 1.

The tubular receptacle 34 has a socket end 343 and an air-permeable end 344 opposite to each other in the longitudinal direction, and a tubular wall segment 341 interposed therebetween to confine a flashback chamber 342. The tubular receptacle 34 is retained in the accommodation compartment 31 by a friction force that is generated between protrusion and recess portions 3411, 323, with the socket end 343 and the air-permeable end 344 respectively confronting the rear plug portion 222 and the rear end wall 322, thereby establishing air communication between the internal duct 225 and the flashback chamber 342. The protrusion portion 3411 abuts against a shoulder 326 formed adjacent to the front end wall 321. An air-permeable member 35 is made from a porous material, and is integrally formed with the air-permeable end 344 so as to prevent flashback blood from trickling out of the flashback chamber 342.

The plunger 3 has two outlets 33 which communicate the accommodation compartment 31 with the ambient air, and which are disposed downstream of the air permeable end 344. In this embodiment, the outlets 33 are formed in the intermediate surrounding wall 32 adjacent to the rear end wall 322 to facilitate closing by an operator's fingers when the operator grips and moves the tubular plunger 3.

The coil spring 26 is disposed within the smaller-diameter portion 124 to surround the needle cannula 23, and is compressed between the forward opening 122 and the front hub portion 291 of the needle seat 29 so as to provide a prodding force for moving the needle seat 29 rearwardly.

The catheter hub 24 defines therein a duct that permits extension of the needle cannula 23 therethrough.

The tubular catheter 25 has a proximate segment which is inserted into the duct, and a distal segment which extends from the proximate segment and which surrounds and sheathes the needle cannula 23 while permitting the tip end 232 of the needle cannula 23 to project forwardly of the distal segment for piercing a patient's skin.

The front protector 14 is sleeved on the second step segment 129 to surround the smaller-diameter portion 124 for shielding the needle cannula 23. The rear protector 15 is sleeved on the sleeved portion 131 to surround the larger-diameter portion 123 for shielding the plunger 3.

A front sealing strip 16 is disposed to peelably adhere the front protector 14 to the larger-diameter portion 123 adjacent to the smaller-diameter portion 124. Specifically, the front sealing strip 16 includes small front and rear adhesive layers 162 which are adhered to the front protector 14 and the larger-diameter portion 123, respectively, and a film layer 161 which is peelably attached to the front and rear adhesive layers 162 and which is formed with a weakened line 163 such that, when the front protector 14 is turned to break the film layer 161 along the weakened line 163, only the rear adhesive layer 162 and a portion of the film layer 161 will remain on the larger-diameter portion 123, thereby permitting viewing inside of the larger-diameter portion 123.

The rear protector 15 is detachably sleeved on the surrounding segment 13. A rear sealing strip 17 is disposed to peelably adhere the rear protector 15 to the surrounding segment 13.

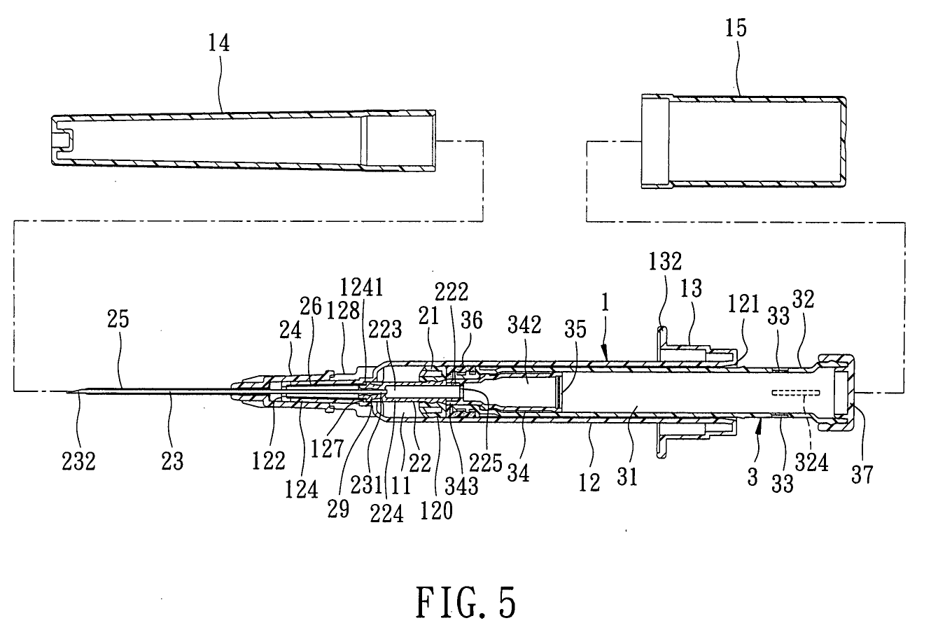

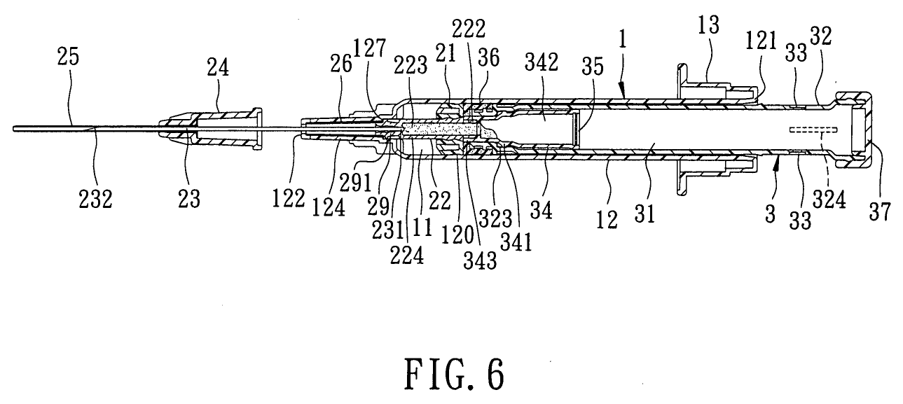

Referring to FIGS. 5 and 6, in an IV introducing stroke, the front and rear protectors 14, 15 are removed first to expose the needle cannula 25 and the plunger 3. The plunger 3 is moved forwardly until the annular sealing member 36 abuts against the grip member 21 and the socket end 343 is engaged with the rear plug portion 222, thereby permitting the front hub portion 291 of the needle seat 29 to be placed in a pre-disposal position. Subsequently, the operator holds the surrounding barrel wall 12 of the barrel 1 with his/her thumb and index fingers to pierce the patient's vein with the tip end 232 so as to introduce the tubular catheter 25 into the vein. Since the pre-chamber 223 is directly communicated with the needle cannula 23, the flow of blood through the pre-chamber 223 into the flashback chamber 342 is visible immediately from the pre-chamber 223 so that the operator can check whether the needle cannula 23 has been inserted properly into the vein. Thereafter, the operator can separate the catheter hub 24 from the barrel 1. The IV introducing operation is thus completed.

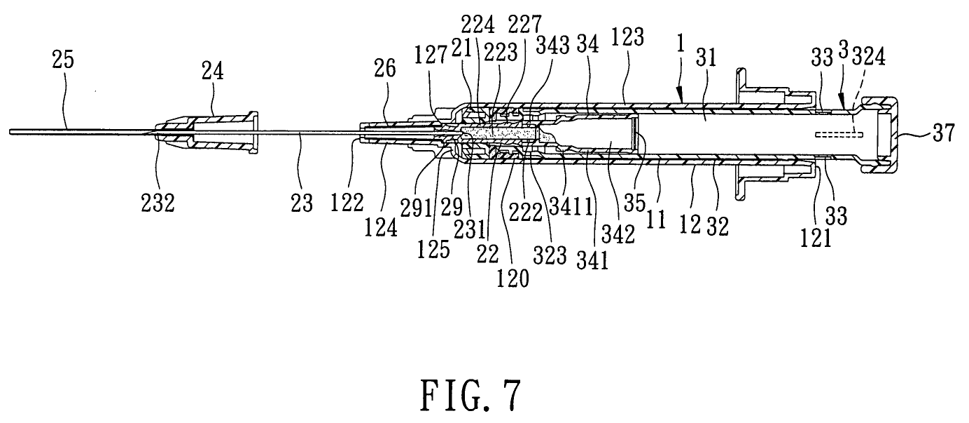

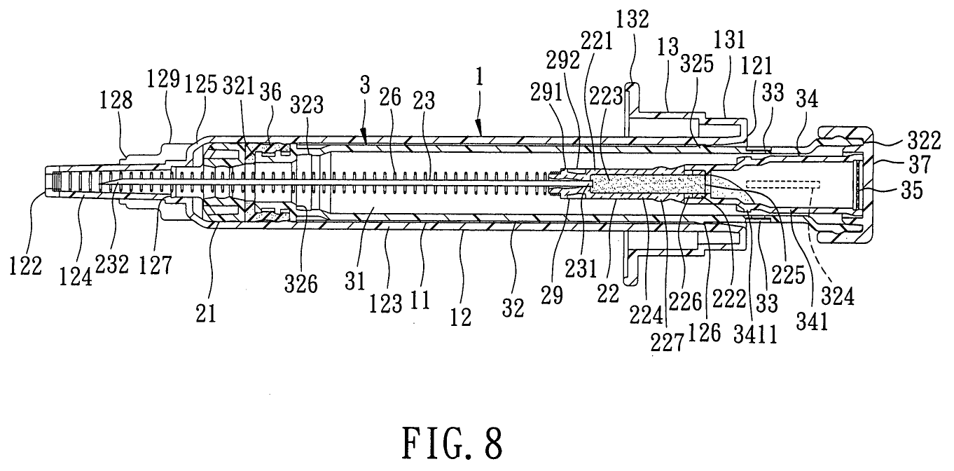

Referring to FIGS. 7 and 8, since the flashback chamber 342 is in air communication with the accommodation compartment 31 so as to be communicated with the ambient air through the outlets 33, after the IV introducing stroke, by subsequently pressing the plunger 3 forwardly, air in the barrel 1 and the plunger 3 can be discharged through the outlets 33 such that the blood can flow into the flashback chamber 342 due to air communication of the flashback chamber 342 with the ambient air, and the air-permeable member 35 can prevent the flashback blood from trickling out of the flashback chamber 342. Thus, the flashback blood will not be forced out of the barrel 1 during forward pressing of the plunger 3.

When the grip member 21 is pushed forward by virtue of forward movement of the front end wall 321 against the holding force, the grip member 21 is moved to disengage from the retaining area 120, and the front hub portion 291 is moved past the friction diminishing area 1241 to abut against the inner abutment surface 127, thereby shifting static frictions between the needle seat 29 and the smaller-diameter portion 124 and between the grip member 21 and the retaining area 120 to kinetic frictions. This facilitates a triggering action for retraction of the needle cannula 24 (to be described in detail hereinafter). Specifically, a material-sticking action and the engagement between the grip member 21 and the retaining area 120 can be minimized at this stage, so that the operator can push the plunger 3 effortlessly.

The needle seat 29, the tubular member 22 and the tubular receptacle 34 remain unmoved due to the abutment of the front hub portion 291 against the inner abutment surface 127 such that, once the plunger 3 is pressed further forward to cause movement of the plunger 3 relative to the tubular receptacle 34 against the friction force, the protrusion portion 3411 of the receptacle 34 is disengaged from the recess portion 323 in the plunger 3 so as to permit releasing of the coil spring 26. Also, the air in the barrel 1 can be released through the accommodation compartment 31 and be discharged from the outlets 33.

Therefore, as shown in FIG. 8, through the engagement of the socket end 343 with the rear plug portion 222, the needle seat 29, together with the tubular receptacle 34, will be moved towards the rear end wall 322 by virtue of a prodding force of the coil spring 26 so as to place the needle seat 29 and the needle cannula 23 in a disposal position, where the tip end 232 of the needle cannula 23 is retracted into the passage 11, and where the socket end 343 is closer to the rear end wall 322 than in the position of use. Thereafter, the plunger 3 is retained at the larger-diameter portion 123 through snug engagement between an annular recess 126 formed in the inner surface of the larger-diameter portion 123, and an annular projection 325 formed on the outer surface of the intermediate surrounding wall 132, so as to prevent rearward pulling of the plunger 3, for safety disposal.

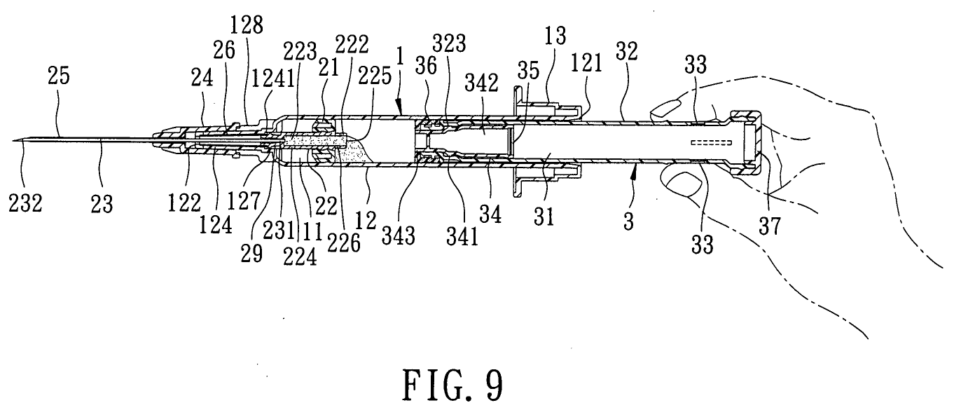

Referring to FIG. 9, during the IV catheter introducing operation, if flashback blood is not observed in the pre-chamber 223 and the flashback chamber 342, the operator can hold the barrel 1 with one hand and the plunger 3 with the other hand, and close the outlets 33 with his/her thumb and index fingers to interrupt air communication between the passage 11 and the ambient air. Then the operator can pull the plunger 3 rearwardly so as to generate a reduced pressure in the passage 11 to thereby facilitate flow of blood into the pre-chamber 223 and the passage 11. Thus, the operator can easily check whether the tubular catheter 25 has been successfully introduced into a target vein of the patient.

It is noted that, with the provision of the radial inlet 226, the blood flowing into the passage 11 between the front end wall 321 and the grip member 21 can be guided into the internal duct 225 and the flashback chamber 342.

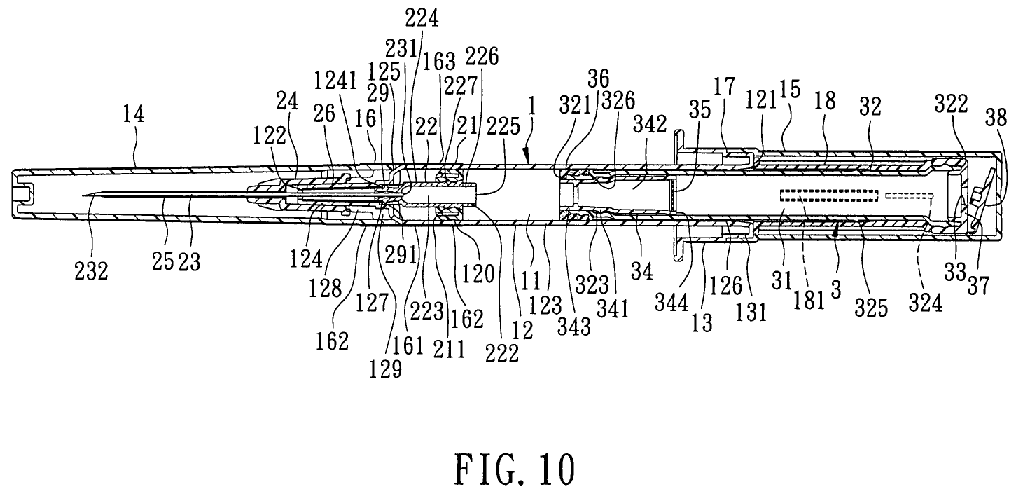

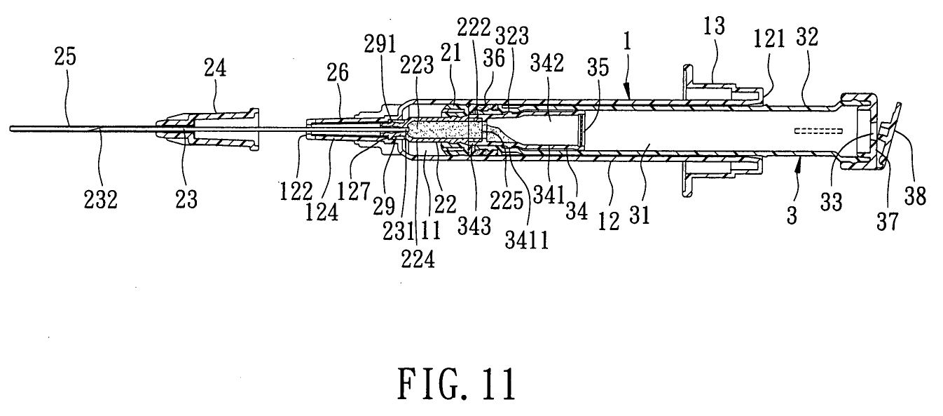

Referring to FIGS. 10 and 11, the second preferred embodiment of an intravenous catheter introducing device according to this invention is shown to be similar to the first preferred embodiment in construction. According to this embodiment, a spacer 18 of a semi-circular shape is detachably disposed between the rearward opening 121 and the end cap 37 in a pre-use position to guard against forward movement of the plunger 3. The spacer 18 has at least one rib 181 extending axially to serve as a handgrip. Additionally, the pre-chamber 223 has a volume which is greater than that of the first preferred embodiment so that the flashback blood flows faster. Further, the outlet 33 is formed in the end cap 37 instead of in the intermediate surrounding wall 32 of the plunger 3. A cover plate 38 is disposed on the end cap 37 and is movable relative to the plunger 3 between a closing position, where the cover plate 38 engages the end cap 37 to close the outlet 33, and an opening position, where the cover plate 38 disengages from the end cap 37 to open the outlet 33.

In an IV catheter introducing stroke, the front and rear protectors 14, 15 and the spacer 18 are removed, and the plunger 3 is subsequently pressed forward to bring the receptacle 34 into engagement with the tubular member 22. Thus, the operator can hold the surrounding barrel wall 12 of the barrel 1 and pierce the patient's vein with one hand.

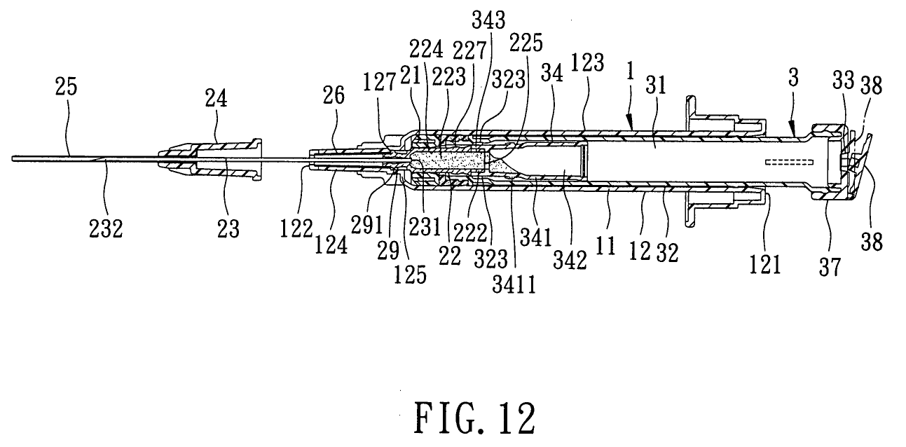

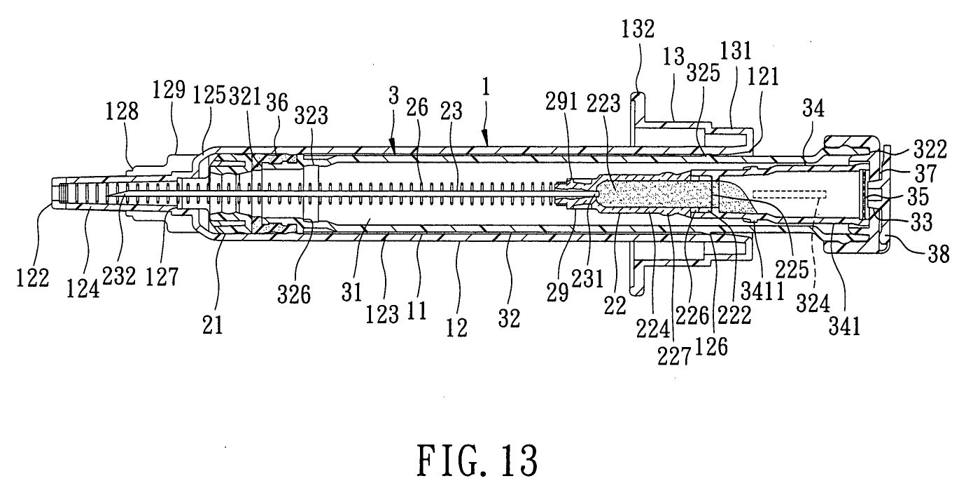

Subsequently, referring to FIGS. 12 and 13, as in the first preferred embodiment, a continued forward pressing of the plunger 3 results in disengagement of the grip member 21 from the gripped segment 227 of the tubular member 22, and disengagement of the receptacle 34 from the intermediate surrounding wall 32 of the plunger 3 such that the needle cannula 23, together with the needle seat 29, the tubular member 22 and the receptacle 34, can be moved to the disposal position.

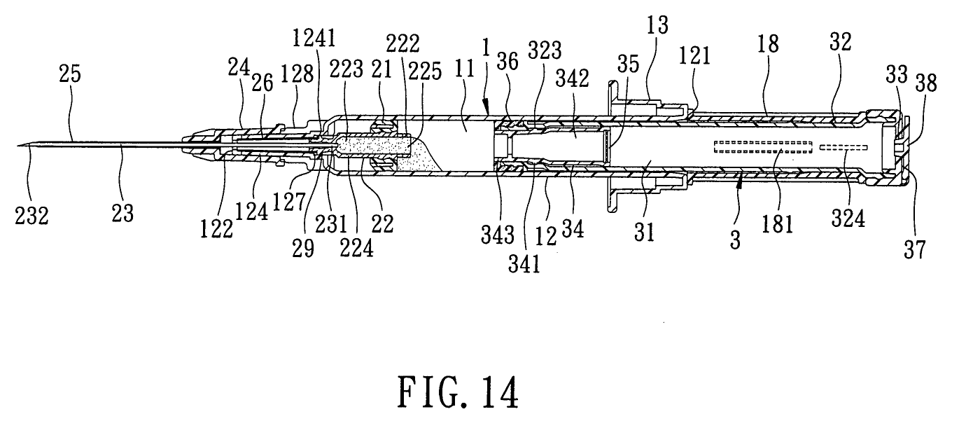

As shown in FIG. 14, if blood is not observed in the pre-chamber 223 and the flashback chamber 342, the operator can move the cover plate 38 to the closing position and hold the barrel 1 (where the tip end 232 may be left inside the vein) with one hand, and pull the plunger 3 rearwardly with the other hand so as to generate a reduced pressure in the passage 11 to thereby facilitate flow of blood into the passage 11. At the same time, the spacer 18 can be once again disposed between the rearward opening 121 and the end cap 37 to maintain the reduced pressure without application of any manual force so as to facilitate the operator to probe the target vein with the tip end 232.

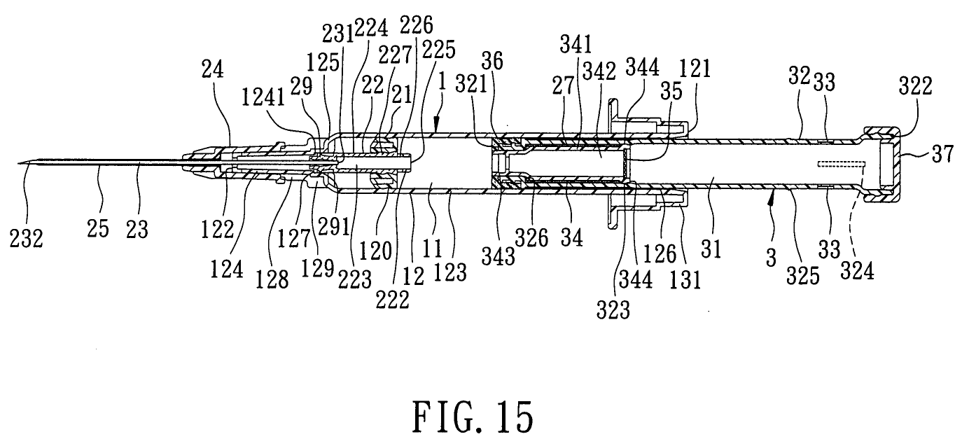

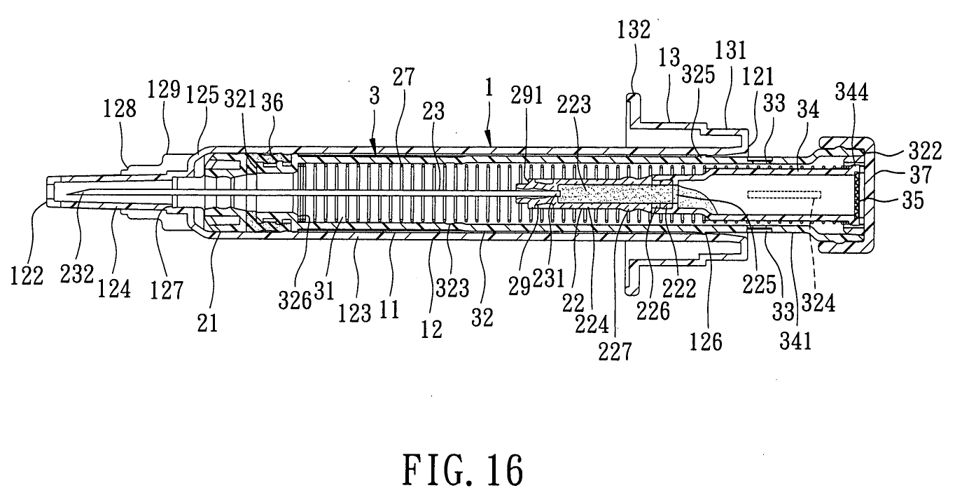

Referring to FIGS. 15 and 16, the third preferred embodiment of an intravenous catheter introducing device according to this invention is shown to be similar to the first preferred embodiment in construction. The difference resides in that the coil spring 27 is received in the accommodation compartment 31, which surrounds the tubular wall segment 341 of the tubular receptacle 34, and which is compressed between the shoulder 326 and the air-permeable end 344.

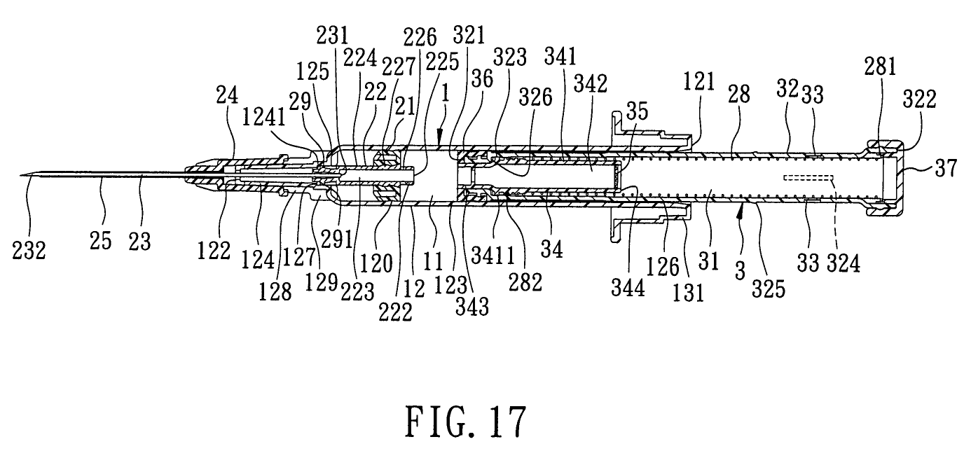

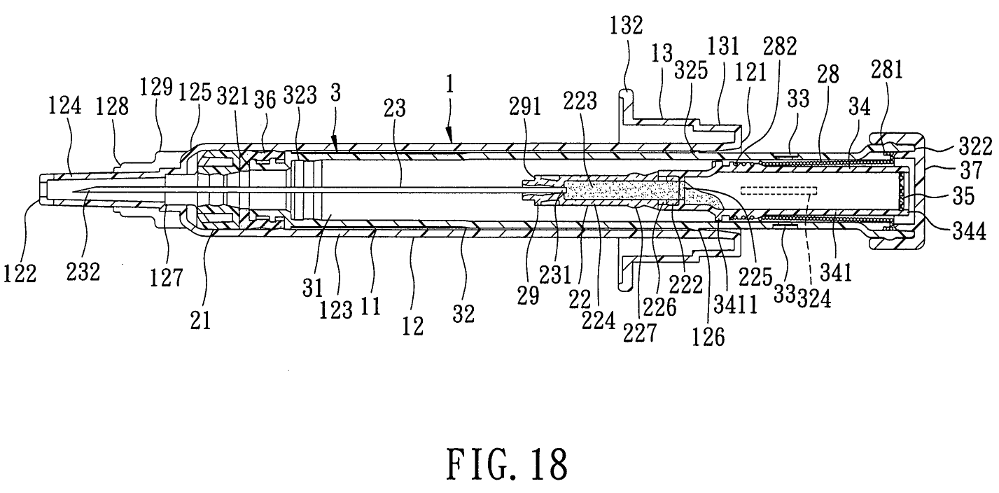

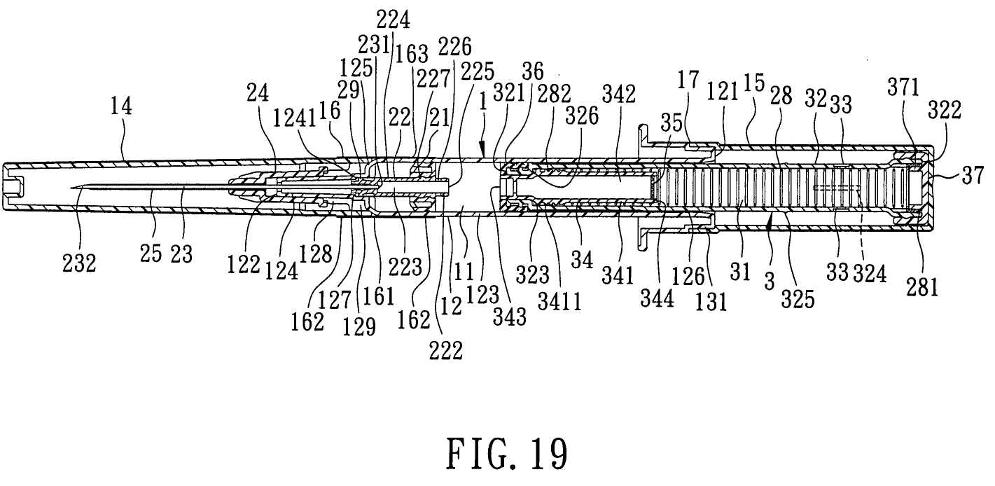

Referring to FIGS. 17 and 18, the fourth preferred embodiment of an intravenous catheter introducing device according to this invention is shown to be similar to the first preferred embodiment in construction. The difference resides in that the coil spring 28 is received in the accommodation compartment 31, and has a secured end 281 which is secured to the intermediate surrounding wall 32 adjacent to the rear end wall 322, and a tensed end 282 which is secured to the tubular wall segment 341 of the tubular receptacle 34 and which remains tensed in the position of use. Alternatively, as shown in FIG. 19, the secured end 281 may be secured to an annular segment 371 of the end cap 37.

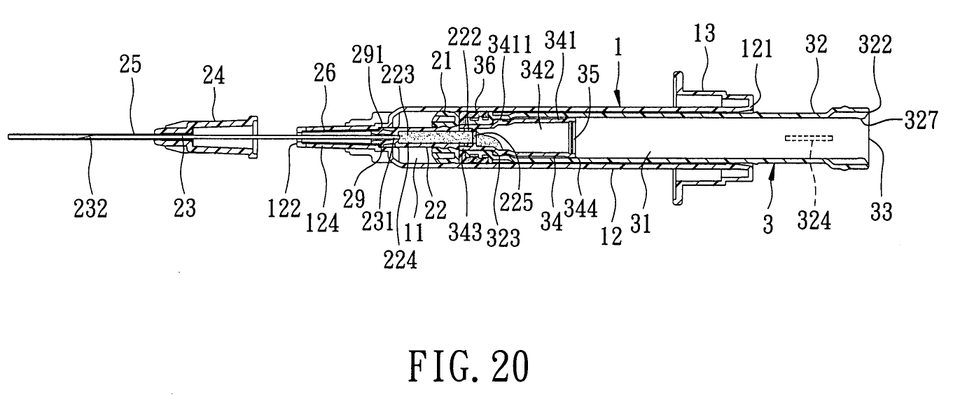

Referring to FIG. 20, the fifth preferred embodiment of an intravenous catheter introducing device according to this invention is shown to be similar to the first preferred embodiment in construction. The difference resides in that an outlet 33 is formed in the rear end wall 322 of the tubular plunger 3 to facilitate closing of the outlet 33 by the operator, and the intermediate surrounding wall 32 of the tubular plunger 3 has a plurality of annular barriers 327 formed adjacent to the rear end wall 322 such that the air-permeable end 344 of the tubular receptacle 34 is stopped by the annular barriers 327 when the tubular receptacle 34 is moved to the disposal position.

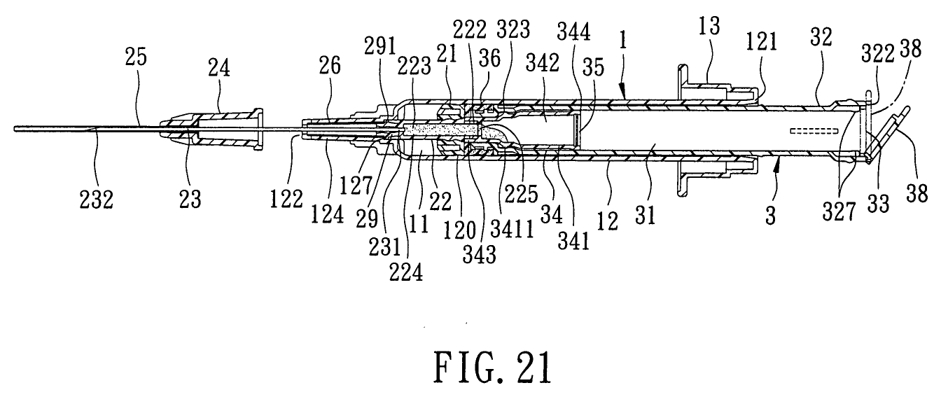

Referring to FIG. 21, the sixth preferred embodiment of an intravenous catheter introducing device according to this invention is shown to be similar to the first preferred embodiment in construction. The difference resides in that an outlet 33 is formed in the rear end wall 322 of the tubular plunger 3 to facilitate closing of the outlet 33 by the operator, and a cover plate 38 is further disposed on the tubular plunger 3 and is movable relative to the tubular plunger 3 between a closing position, where the cover plate 38 engages the rear end wall 322 to close the outlet 33, and an opening position, where the cover plate 38 disengages from the rear end wall 322 to open the outlet 33.

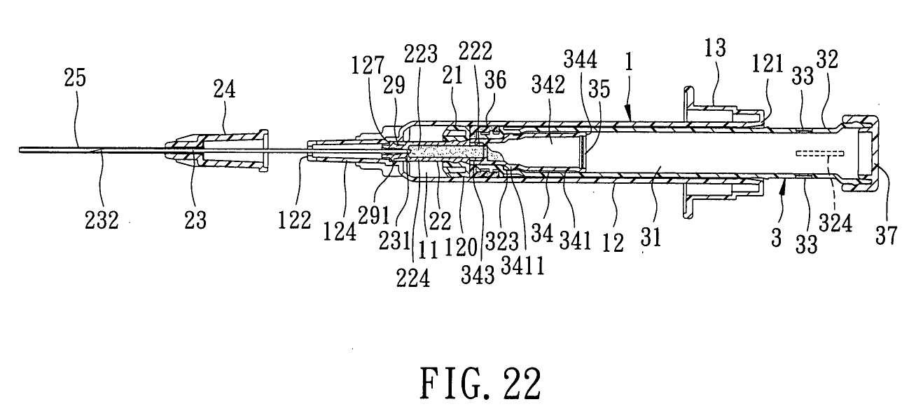

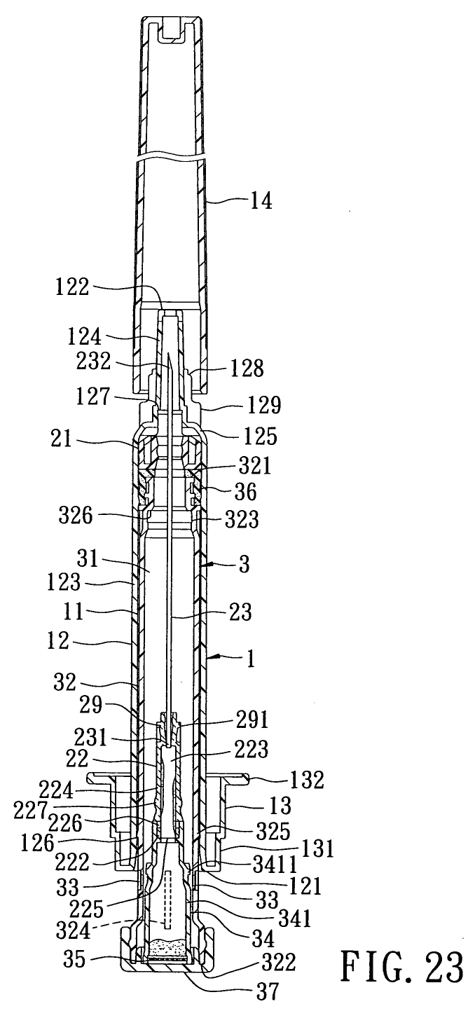

Referring to FIGS. 22 and 23, the seventh preferred embodiment of an intravenous catheter introducing device according to this invention is shown to be similar to the first preferred embodiment in construction. The difference resides in that the coil spring 26 is eliminated. Hence, when the grip member 21 is disengaged from the gripped segment 227 of the tubular member 22, and the receptacle 34 is disengaged from the intermediate surrounding wall 32 of the plunger 3 by virtue of a continued forward pressing of the plunger 3, the operator can place the barrel 1 in an upright position, i.e., the forward opening 122 is up and the rearward opening 121 is down, such that the needle cannula 23, together with the needle seat 29, the tubular member 22 and the receptacle 34, can be moved to the disposal position by gravity. Thereafter, the front protector 14 can be sleeved on the second step segment 129 to close the barrel 1.

As illustrated, according to the intravenous catheter introducing device of this invention, the used needle cannula 23 can be retracted into the plunger 3 to thereby avoid occurrence of an accidental needle stick. Further, during the forward pressing of the plunger 3 to retract the needle cannula 23, since the pre-chamber 223 is air communicated with the ambient air, the flashback blood may flow into the pre-chamber 223 and the flashback chamber 342, and the air-permeable member 35 can prevent the flashback blood from trickling out. Thus, blood will not be forced out of the barrel 1.

Moreover, during the IV catheter introducing operation, once flashback blood is not observed in the pre-chamber 223, the operator can close the outlet(s) 33 and pull the plunger 3 rearwardly to generate a reduced pressure in the passage 11 to thereby facilitate flow of indicator blood into the passage 11. Thus, the operator can easily check whether the tubular catheter 25 has been successfully introduced into a target vein of the patient. Furthermore, the indicator blood flow is visible immediately through the pre-chamber 223 so as to shorten the time needed for the IV catheter introducing operation. By providing the front and rear protectors 14, 15 and the front and rear sealing strips 16, the air and water tightness of the device of the present invention can be maintained, and packing cost can be reduced.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

We claim:

1. An intravenous catheter introducing device comprising:
a barrel having a surrounding barrel wall which surrounds an axis in a longitudinal direction, and which defines a passage therein, said passage having rearward and forward openings which are opposite to each other in the longitudinal direction, said surrounding barrel wall including a larger-diameter portion and a smaller-diameter portion which are disposed proximate to said rearward and forward openings, respectively, said smaller-diameter portion having an inner abutment surface which faces rearwards, said larger-diameter portion having a retaining area which is spaced apart from said smaller-diameter portion in the longitudinal direction;
a needle cannula having a tip end and a rear connecting end opposite to each other along the axis in the longitudinal direction;
a tubular needle seat including a front hub portion which confronts said inner abutment surface so as to be engageable therewith, and a rear fitting portion which is opposite to said front hub portion in the longitudinal direction, and which is configured to fix said rear connecting end;
a light-transmissible hollow tubular member which includes a front open end formed with said rear fitting portion, and in fluid communication with said rear connecting end, and tubular monitoring and gripped segments that are proximate to and distal from said front open end, respectively, said tubular monitoring segment defining a pre-chamber for flashback blood, said tubular member further including a rear plug portion which extends from said gripped segment, which is distal from said front open end, and which has an internal duct extending therethrough to be communicated with said pre-chamber;
a tubular grip member which, in a position of use, is disposed to hold, with a holding force, said gripped segment in a position of immovability along the axis relative to said retaining area;
a tubular plunger which is disposed to be movable in said passage along said larger-diameter portion, said plunger having a front end wall which is movable to abut against said grip member, a rear end wall which is disposed opposite to said front end wall, and which extends outwardly of said rearward opening so as to be manually operable, and an intermediate surrounding wall which is interposed between said front and rear end walls, and which defines an accommodation compartment;
a tubular receptacle which has a socket end and an air-permeable end opposite to each other in the longitudinal direction, and a tubular wall segment interposed therebetween to confine a flashback chamber, said tubular receptacle being disposed to be retained in said accommodation compartment by a friction force, with said socket end and said air-permeable end respectively confronting said rear plug portion and said rear end wall, thereby establishing air communication between said internal duct and said flashback chamber,
such that, when said front end wall is brought to abut against said grip member, said socket end is engaged with said rear plug portion to thereby permit said front hub portion to be placed in a pre-disposal position,
such that, when said grip member is pushed forward by virtue of forward movement of said front end wall against the holding force, the pre-disposal position of said front hub portion facilitates release of said gripped segment from said grip member, thereby permitting axial and rearward movement of said gripped segment relative to said retaining area, and
such that continued forward movement of said front end wall against the friction force results in movement of said needle seat, and movement of said tubular receptacle together therewith through the engagement of said rear plug portion with said socket end, towards said rear end wall by virtue of a prodding force so as to place said needle seat and said needle cannula in a disposal position, where said tip end of said needle cannula is retracted into said passage, and where said socket end is closer to said rear end wall than in the position of use.

2. The intravenous catheter introducing device according to claim 1, wherein said hollow tubular member is integrally formed with said tubular needle seat, and is made from a light transmissible material.

3. The intravenous catheter introducing device according to claim 1, further comprising an air-permeable member which is made from a porous material, and which is engaged with said air-permeable end so as to prevent flashback blood from trickling out of said flashback chamber.

4. The intravenous catheter introducing device according to claim 1, wherein said tubular plunger has a deformable annular sealing member which is configured to surround said front end wall, and which is in air-tight sliding engagement with said larger-diameter portion.

5. The intravenous catheter introducing device according to claim 1, wherein said tubular plunger has at least one outlet that communicates said accommodation compartment with the ambient air, and that is disposed downstream of said air permeable end.

6. The intravenous catheter introducing device according to claim 5, wherein said outlet is formed in said intermediate surrounding wall adjacent to said rear end wall to facilitate closing by a user's finger when the user grips and moves said tubular plunger.

7. The intravenous catheter introducing device according to claim 5, wherein said rear end wall of said tubular plunger defines an access opening for insertion of said tubular receptacle into said accommodation compartment, said tubular plunger including an end cap which is detachably mounted to said rear end wall to close said access opening, said outlet being formed in said end cap.

8. The intravenous catheter introducing device according to claim 7, further comprising a cover plate which is disposed on said end cap and which is movable relative to said tubular plunger between a closing position, where said cover plate engages said end cap to close said outlet, and an opening position, where said cover plate disengages from said end cap to open said outlet.

9. The intravenous catheter introducing device according to claim 5, wherein said outlet is formed in said rear end wall of said tubular plunger.

10. The intravenous catheter introducing device according to claim 9, further comprising a cover plate which is disposed on said tubular plunger and which is movable relative to said tubular plunger between a closing position, where said cover plate engages said rear end wall to close said outlet, and an opening position, where said cover plate disengages from said rear end wall to open said outlet.

11. The intravenous catheter introducing device according to claim 1, further comprising a coil spring which is disposed within said smaller-diameter portion, which surrounds said needle cannula, and which is compressed between said forward opening and said front hub portion of said needle seat so as to provide the prodding force.

12. The intravenous catheter introducing device according to claim 1, further comprising a coil spring which is received in said accommodation compartment, which surrounds said tubular wall segment, and which is compressed between said front end wall and said air-permeable end so as to provide the prodding force.

13. The intravenous catheter introducing device according to claim 1, further comprising a coil spring which is received in said accommodation compartment, and which has a secured end that is secured to said intermediate surrounding wall adjacent to said rear end wall, and a tensed end that is secured to said tubular wall segment of said tubular receptacle to remain tensed in the position of use so as to provide the prodding force.

14. The intravenous catheter introducing device according to claim 1, wherein said smaller-diameter portion has a friction diminishing area which is disposed between said inner abutment surface and said front hub portion such that, when said grip member is pushed forward by virtue of the forward movement of said front end wall against the holding force, said front hub portion is moved past said friction diminishing area to abut against said inner abutment surface to thereby facilitate effectuation of the prodding force for retraction of said needle cannula.

15. The intravenous catheter introducing device according to claim 1, further comprising front and rear protectors which are disposed to surround said smaller-diameter portion and said larger-diameter portion, respectively, for shielding said needle cannula and said plunger, respectively.

16. The intravenous catheter introducing device according to claim 15, further comprising a front sealing strip which is disposed to peelably adhere said front protector to said larger-diameter portion adjacent to said smaller-diameter portion.

17. The intravenous catheter introducing device according to claim 15, wherein said barrel has a surrounding segment which extends forwardly from said rearward opening to terminate at a finger flange, and which surrounds said larger-diameter portion, said rear protector being detachably sleeved on said surrounding segment, said device further comprising a rear sealing strip which is peelably adhere said rear protector to said surrounding segment.

18. The intravenous catheter introducing device according to claim 1, wherein said rear plug portion of said tubular member is disposed rearwardly and outwardly of said grip member, and has a radial inlet which extends radially to communicate said passage with said pre-chamber.

19. The intravenous catheter introducing device according to claim 1, further comprising a spacer which is detachably disposed between said rearward opening and said rear end wall in a pre-use position to thereby guard against a forward movement of said plunger.
